(19) United States
(12) Patent Application Publication
(10) Pub. No.: US 2022/0403258 A1
(43) Pub. Date: Dec. 22, 2022

(54) METHODS AND SYSTEMS FOR PARAFFIN ISOMERIZATION OPTIMIZATION

(71) Applicant: ExxonMobil Technology and Engineering Company, Annandale, NJ (US)

(72) Inventors: Xinrui Yu, Furlong, PA (US); Jihad M. Dakka, Whitehouse Station, NJ (US); Guang Cao, Princeton, NJ (US); Wesley Sattler, Parsippany, NJ (US)

(21) Appl. No.: 17/807,019

(22) Filed: Jun. 15, 2022

Related U.S. Application Data
(60) Provisional application No. 63/202,587, filed on Jun. 17, 2021.

(51) Int. Cl. C10G 49/04; B01J 23/30; B01J 23/00

(52) U.S. Cl. CPC C10G 49/04; B01J 23/30; B01J 23/002; C10G 2300/1085; B01J 2523/48; B01J 2523/68

(57) ABSTRACT

Systems and methods for producing an isomerization product. One or more isomerization reactors comprising a catalyst may be used to process an isomerization feedstock comprising a primary n-paraffin reactant and hydrogen gas, and the isomerization reactor may be operated at a pressure parameter at which the partial pressure of the primary n-paraffin is within about 70% to about 130% of its equilibrium vapor pressure to isomerize the primary n-paraffin reactant.

### CROSS REFERENCE TO RELATED APPLICATIONS

This application claims priority to and the benefit of U.S. Ser. No. 63/202,587 filed on Jun. 17, 2021, the entire contents of which are incorporated herein by reference.

### FIELD OF THE INVENTION

The present disclosure relates to methods and systems for paraffin isomerization optimization and, more particularly, to methods and systems for optimizing paraffin isomerization processing to increase isomer conversion rates based on a pressure parameter.

### BACKGROUND OF THE INVENTION

Paraffin isomerization to higher branched alkanes typically occurs via bifunctional catalysis, meaning that the paraffin molecules dehydrogenate over metal sites on a catalyst, forming intermediate olefins that become protonated at acid sites to form carbenium ions. The carbenium ions can undergo skeletal isomerization through paths such as the cyclopropyl cation mechanism.

In a typical paraffin isomerization process, a paraffin feedstock is mixed with hydrogen and heated to an isomerization reactor temperature. The mixture is passed over the metal catalyst in one or more isomerization reactors, where n-paraffins are catalytically isomerized to branched isomers. The product is subsequently separated in a product separator into two streams, one comprising the isomer product and the other comprising recycled hydrogen.

Paraffin isomerization can take place at temperatures between about 100° C. and about 300° C., where lower temperatures select for thermodynamically favored branched isomers having a higher octane rating. Higher octane rating is generally desirable in the petrochemical and other industries, such as for providing valuable blending components for the manufacture of premium gasolines. However, industrial scale commercialization requires a tradeoff between desired branched isomers and economic limitations, among other manufacturing constraints, such as facility footprint, equipment processing capacity, and the like. Traditionally, this balance is met by either accelerating isomerization conversion rate, thereby resulting in higher octane rating, achieved by increasing the temperature of the isomerization reaction, or upscaling facility size and equipment to permit longer residence times at low temperatures.

### SUMMARY OF THE INVENTION

The present disclosure relates to methods and systems for paraffin isomerization optimization and, more particularly, to methods and systems for optimizing paraffin isomerization processing to increase isomer conversion rates based on a pressure parameter.

According to one or more aspects of the present disclosure, provided herein is a method including processing an isomerization feedstock comprising a primary n-paraffin reactant and hydrogen gas in an isomerization reactor comprising a catalyst. The isomerization reactor may be operated at a pressure parameter at which the partial pressure of the primary n-paraffin is within about 70% to about 130% of its equilibrium vapor pressure to isomerize the primary n-paraffin reactant. According to one or more aspects of the present disclosure, provided herein is a system including an isomerization reactor and a first separator. The isomerization reactor may include a catalyst for receiving an isomerization feedstock comprising a primary n-paraffin reactant and hydrogen gas, wherein the isomerization reactor may be operated at a pressure parameter at which the partial pressure of the primary n-paraffin is within about 85% to about 115% of its equilibrium vapor pressure. The first separator may be used for separating isomerization product and recycled hydrogen gas.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates three representative T-P phase diagram curves for n-C5, n-C6, and n-C7.

FIG. 2 illustrates representative isomers of n-C7 which may be generated during an isomerization process.

FIG. 3 illustrates a representative system for implementing an isomerization method, according to one or more aspects of the present disclosure.

FIG. 4 illustrates plot showing overall n-C7 conversion against the initial partial pressures of n-C7, according to one or more aspects of the present disclosure.

FIG. 5 illustrates RON and MON numbers for various C7 isomers, according to one or more aspects of the present disclosure.

FIG. 6 illustrates RON and MON numbers of C7 paraffin products at different initial partial pressures for n-C7.

### DETAILED DESCRIPTION OF THE INVENTION

The present disclosure relates to methods and systems for paraffin isomerization optimization and, more particularly, to methods and systems for optimizing paraffin isomerization processing to increase isomer conversion rates based on a pressure parameter.

As described above, higher octane rating is generally desirable in the petrochemical and other industries, such as for providing valuable blending components for the manufacture of premium gasolines. Yet, industrial scale commercialization requires a tradeoff between desired branched isomers and other practical limitations, traditionally achieved by increasing the temperature of the isomerization reaction or increasing the residence time of the isomerization reaction. However, higher paraffin isomerization reaction temperatures can prompt side reactions, such as cracking, resulting in liquid yield loss; longer residence times often require decreased throughput and/or larger reactor sizes, increasing capital expenditures.

The present disclosure provides methods and systems for increasing conversion rates to obtain highly desirable branched paraffin isomerization products having high octane values by capitalizing on process parameters. More specifically, different than traditional techniques, the methods and systems of the present disclosure identify the vapor pressure of an n-paraffin feedstock for use in a paraffin isomerization process to maximize isomerization conversion rate and increase branched isomer products thereof based on a pressure parameter, thereby avoiding traditional use of higher reaction temperatures or higher residence processing strategies.

As used herein, the term “n-paraffin,” and grammatical variants thereof, refers to normal, linear (straight chain) hydrocarbons having a carbon number of C4, C5, C6, or C7. The term “n-paraffin” with reference to the various feedstocks described herein is not intended to limit said feedstock to only n-paraffins; rather, the feedstocks may contain one or more other constituents, without limitation, such as aromatics, naphthenes, isoparaffins, contaminates (e.g., compounds containing sulfur, nitrogen, metals, and the like), impurities (e.g., oxygenate, and the like), and the like, without departing from the scope of the present disclosure; however, the primary constituent is one or more n-paraffins as defined herein.

As used herein, the term “feedstock,” and grammatical variants thereof, refers to the raw material supply for an isomerization process comprising one or more n-paraffins, natural or synthetic. The feedstocks of the present disclosure comprise a primary reactant, defined as the most abundant n-paraffin type (based on carbon number, e.g., n-C4, n-C5, n-C6, n-C7, n-C8, and higher n-C8+) in the feedstock. As used herein, the term “primary reactant,” and grammatical variants thereof, with respect to one or more feedstocks described herein, refers to a singular n-paraffin (i.e., single, specific n-paraffin) present in an amount of greater than about 6% by weight (wt. %) of the feedstock, including up to 100%, encompassing any value and subset therebetween, such as in an amount of about 10 wt. % to about 100 wt. % of the feedstock, or about 20 wt. % to about 100 wt. % of the feedstock, encompassing any value and subset therebetween. The terms “primary reactant,” “n-paraffin,” and “primary n-paraffin reactant,” and grammatical variants thereof, may be used interchangeably herein.

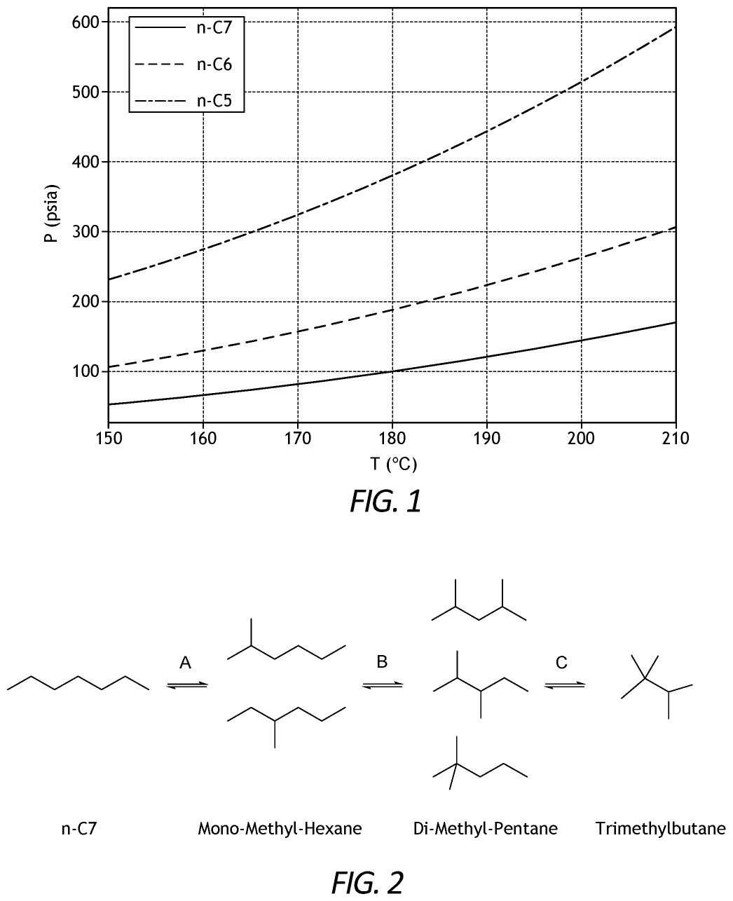

As used herein, the term “vapor pressure,” and grammatical variants thereof, refers to the pressure at which the vapor phase of a material (i.e., an n-paraffin feedstock) is at phase equilibrium with its liquid phase at a given temperature. The vapor pressure may be determined using a temperature-pressure (T-P) phase diagram curve for a given n-paraffin feedstock concentration, and may also be referred to as point of gas-liquid transition phase. For a mixture of multiple material types, the partial pressure of each material is its molar fraction in vapor phase multiplied by the total pressure. FIG. 1 illustrates three representative T-P phase diagram curves for n-C5, n-C6, and n-C7, in which temperature (T) is provided in ° C. along the x-axis and vapor pressure (P) is provided in pounds per square inch absolute (psia) along the y-axis. As shown, as temperature decreases, favoring higher conversion rates to more branched isomers in an isomerization reaction, vapor pressure actually decreases at the corresponding temperature. Conventional understanding of paraffin isomerization on solid catalyst is that isomerization performed at higher pressures prompts increased or constant conversion rate; however, as provided herein, it is demonstrated that when the pressure is increased above the vapor pressure of primary n-paraffin, conversion rates actually decrease.

It is to be understood that the T-P curves of FIG. 1 are merely representative and not intended to be limiting in terms of temperature range or pressure range shown or in terms of the particular n-paraffins depicted.

Moreover, the partial pressure of a particular material is dependent on the molar fraction availability of said material in vapor phase and hydrogen ratio. As the mole fraction of the primary reactant decreases as isomerization proceeds, the required total pressure at which the n-paraffin partial pressure reaches its phase equilibrium vapor pressure increases as isomerization temperature is held constant. That is, in order to reach or maintain the vapor pressure for the primary reactant (n-paraffin), the total pressure is increased to compensate for the decrease of its molar fraction as isomerization proceeds.

During the isomerization process, an n-paraffin hydrocarbon feedstock is processed and converted into isomers that have the same carbon number but are more highly branched than the initial feedstock. These more highly branched isomers exhibit higher octane numbers compared to the n-paraffin feedstock. FIG. 2 illustrates three representative isomers of normal-heptane (n-C7)—mono-methyl-hexane, di-methyl-pentane, and trimethylbutane—which may be generated during an isomerization process. Referring to FIG. 2, during the isomerization process, n-C7 may reversibly react (A) to form isomers mono-methyl-hexane; mono-methyl-hexane may reversibly react (B) to form isomers di-methyl-pentane; and di-methyl-pentane may reversibly react (C) to form isomer tri-methyl-butane. The process or reaction parameters of the isomerization process controls the reversibility, or cracking, of the branched or higher branched isomers into unbranched n-paraffins or less branched isomers thereof. The conversion of each of the reactions described above are reversible and they are limited by thermodynamic equilibrium, which is determined by reaction conditions. The process control logic described hereinbelow in greater detail is designed to push the conversion close to equilibrium as quickly as possible to maximize high branched isomers and therefore to maximize the octane number.

It is to be appreciated that FIG. 2 is merely illustrative of representative n-C7 isomers, and, moreover, other n-paraffins (e.g., n-C4-n-C6, n-C8, and the like) will have branched isomers corresponding to their carbon number and similar isomerization mechanism and reversibility.

As isomeric branching increases, octane number increases. Octane number is generally determined using one of two methods: (1) the research octane number (RON) method or (2) the motor octane number (MON) method, which differ principally in specific testing conditions. Generally, isomerization branched isomer products have an RON and/or MON value in the range of about 40 to about 120, such as in the range of a lower limit of about 40, 45, 50, 55, 60, 65, 70, or 75 to an upper limit of about 120, 115, 110, 95, 90, 85, or 80, encompassing any value and subset therebetween.

The methods and systems described herein relate to isomerization based on a pressure parameter at any given temperature; the pressure parameter is selected based on a pressure value at or near the vapor pressure for the primary n-paraffin feedstock constituent for performing isomerization, at which the isomerization conversion rate is observed to peak as further described hereinbelow. As used herein, the term “pressure parameter,” and grammatical variants thereof, refers to a pressure at which the partial pressure of the primary reactant (n-paraffin) is within about 70% to about 130% of its equilibrium vapor pressure within a feedstock (based on the temperature of isomerization and the available molar ratio of the primary n-paraffin reactant for isomerization), including less than or equal to about ±10%, or less than or equal to about ±5%, or equal thereto, encompassing any value and subset therebetween.

Without being bound by theory, it is believed that the isomerization rate peaks at or near the phase equilibrium vapor pressure (gas-liquid transition phase) of the primary n-paraffin reactant based on either or both of: (1) at or near the gas-liquid transition phase, formation of at least the primary reactant on or about the catalyst surface is promoted while maintaining diffusion lower than the condensed liquid phase, thus promoting overall adsorption of the primary reactant on catalyst active sites and/or (2) the boiling status on the catalyst surface provides an effective way for isomer products desorption from the catalyst surface. That is, without being bound by theory, the methods and systems described herein are believed to exhibit increased conversion rates and increased branched isomer products using a pressure parameter (compared to traditional isomerization processes that utilize higher temperature or longer residence time) by promoting adsorption of a primary reactant and desorption of formed isomer products at catalyst active sites. It is to be appreciated that other n-paraffin reactants may be present and experience the same or similar “adsorption/desorption control” during the isomerization process, but the pressure parameter is tuned specifically to the primary reactant. Such control logic can be implemented in either batch process or continuous process. In a batch process, pressure can be increased to keep the partial pressure of the primary n-paraffin reactant close to its vapor pressure; in a continuous process, the pressure can be set to keep the initial partial pressure of primary n-paraffin reactant close to its vapor pressure. Multi-stage continuous reactors at different pressures can be used to compensate for the decreased molar fraction of primary n-paraffin reactant (and other n-paraffins, if present) by isomerization conversion.

As described herein, the methods and systems of the present disclosure may be used to increase overall n-paraffin conversion rates to desired branched isomers thereof (see Example below). The overall conversion rate comprises all isomers of the primary n-paraffin reactant, without distinguishing between each branched type. The higher the overall conversion rate of the feedstock material, the higher the isomer content and octane number of the resultant products. In one or more aspects, the present disclosure may achieve overall conversion rates in the range of about 10% to about 100% of an initial n-paraffin feedstock, or about 40% to about 90%, or about 50% to about 90% of an initial n-paraffin feedstock, encompassing any value and subset therebetween. The particular conversion rate may depend on a number of factors including, but not limited to, the initial faction of n-paraffin in the feedstock.

Isomerization temperature(s) (e.g., reactor temperature(s)) of the methods and systems disclosed herein may be in the range of about 120° C. to about 220° C., encompassing any value and subset therebetween, with lower temperatures being preferred, such as those in the range of about 120° C. to about 190° C., or about 150° C. to about 170° C., or about 160° C. to about 170° C., or about 170° C. to about 190° C., encompassing any value and subset therebetween. The selected isomerization temperature may depend on a number of factors including, but not limited to, the particular primary reactant, the configuration of the isomerization processing equipment (e.g., single or multi-stage), the particular catalyst selected, and the like, and any combination thereof.

The hydrogen:hydrocarbon (total hydrocarbon) molar ratio (or “H2:HC ratio”) of the isomerization methods and systems of the present disclosure may be in the range of about 0.5 to about 4.0, encompassing any value and subset therebetween, such as in the range of about 0.5 to about 1.0, or about 1.0 to about 2.0, encompassing any value and subset therebetween. Depending on the hydrogen:hydrocarbon ratio and n-paraffin content, the control logic of the present disclosure provides for adjusting the total pressure to make the partial pressure of n-paraffin close to its vapor pressure. Since isoparaffins have lower boiling points than n-paraffin with the same carbon number, at the isomerization process pressure which the partial pressure of the primary n-paraffin reactant is close to its vapor pressure, other isomers are in the gas phase. As provided herein, the partial pressure of primary n-paraffin reactant within a feedstock at the outset and during isomerization may be calculated using Equation 1 and Equation 2 below:

$$r ( H 2 : HC ) = \frac{m ( {H}_{2} )}{\sum m ( HC )} \qquad Equation 1$$

$$P ( n - paraffin ) = {P}_{t} \cdot \frac{1}{r + 1} \cdot \frac{m ( n - paraffin )}{\sum m ( HC )} , \qquad Equation 2$$

in which r is the molar ratio of hydrogen to hydrocarbon, Pₜ is the total reactor pressure, m(H₂) is the molar flow rate of hydrogen, Σm(HC) is the total molar flow rate of hydrocarbon, m(n-paraffin) is the molar flow rate of primary n-paraffin reactant.

According to various aspects of the present disclosure, the selected pressure parameter (Pₜ) will be based on the vapor pressure of the primary n-paraffin reactant at the selected isomerization temperature, as well as the initial and ongoing molar ratio concentration of primary n-paraffin reactant available for isomerization, and as defined hereinabove, as provided in Equation 3.

$${P}_{t} = {P}_{ν} ( n - paraff in at T ) \cdot ( r + 1 ) \cdot \frac{\sum m ( HC )}{m ( n - paraffin )} , \qquad Equation 3$$

in which Pᵥ(n-paraffin at T) is the vapor pressure of n-paraffin at temperature T. In one or more aspects, the pressure parameter is generally in the range of about 70 psia to about 600 psia, such as about 100 psia to about 500 psia, or about 100 psia to about 400 psia, encompassing any value and subset therebetween.

Table 1 below provides representative examples of n-paraffin isomerization reaction processing parameters for use in one or more aspects of the present disclosure. As shown, the amount of partial pressure of the isomerization reaction for maintaining the primary n-paraffin reactant at the vapor pressure depends on factors such as the amount of primary n-paraffin reactant in the feedstock (at any given time, including as the reaction progresses), the isomerization temperature, and the H2:HC ratio.

TABLE 1

| | Amount of | | Isomeri- | | Isomeri- |
|---|---|---|---|---|---|
| Primary | Primary | | zation | | zation |
| Reactant | Reactant | | Reaction | H2:HC | Reactor |
| Type | in Feedstock | | Temperature | ratio | Pressure |
| n-C7 | 100 | mol % | 180° C. | 1.0 | 199.4 psia |
| n-C7 | 70 | mol % | 180° C. | 1.5 | 356.1 psia |
| n-C7 | 100 | mol % | 170° C. | 2.0 | 246.6 psia |
| n-C7 | 70 | mol % | 170° C. | 1.5 | 293.6 psia |
| n-C6 | 90 | mol % | 180° C. | 1.0 | 414.2 psia |
| n-C6 | 80 | mol % | 170° C. | 1.0 | 391.0 psia |
| n-C5 | 100 | mol % | 160° C. | 0.5 | 411.6 psia |

The isomerization methods and systems described herein are operated at a particular liquid hourly space velocity (LHSV), which is the ratio of volumetric flow rate (hourly) of feedstock to the volume of catalyst. Generally, lower LHSV favors isomerization conversion rates, provided that the selected LHSV does not interfere with liquid distribution or under-utilization of the catalyst. In one or more aspects, the LHSV of the methods and systems of the present disclosure is in the range of about 0.5 hr⁻¹ to about 8.0 hr⁻¹, encompassing any value and subset therebetween, with lower LHSV being preferred, such as those in the range of about 0.5 hr⁻¹ to about 1.0 hr⁻¹, or about 1.0 hr⁻¹ to about 2.0 hr⁻¹, or about 1.0 hr⁻¹ to about 5.0 hr⁻¹, or about 0.5 hr⁻¹ to about 2.0 hr⁻¹, encompassing any value and subset therebetween.

The particular catalyst selected for use in the isomerization methods and systems of the present disclosure is not considered to be particularly limited. In one or more aspects, the catalyst may be a metal selected from the group consisting of a Group IVB (Group 4), a Group VIB metal (Group 6), a Group VIIIB (Groups 8, 9, or 10), a zeolite, a molecular sieve or crystalline microporous material of the MWW framework type, and the like, and any combination thereof. In one or more aspects of the present disclosure, the catalyst is a mixed metal oxide catalyst having an acidic solid oxide component containing an oxide of a Group IVB (Group 4) metal modified with an anion or oxyanion of a Group VIB metal (Group 6). The mixed metal oxide catalysts may be prepared by co-precipitation, followed by calcination and extrusion to form catalyst particles. An example of a suitable mixed metal oxide catalyst includes a Pt impregnated Fe/(WO3)x/(ZrO2)y solid acid catalyst. The molar ratio of y:x can be in the range of 1 to 5, such as 1:1, 1:2, 1:3, 1:4, 1:5, and the like, encompassing any value and subset therebetween. Another example of a suitable mixed metal oxide catalyst includes a Pt impregnated (WO3)x/(ZrO2)y. The molar ratio of y:x can be in the range of 1 to 5, such as 1:1, 1:2, 1:3, 1:4, 1:5, and the like, encompassing any value and subset therebetween. Other suitable mixed metal oxide catalysts include, but are not limited to, in U.S. Pat. Nos. 5,902,767, 6,706,659, 7,399,896, and 2003/0069131, the entireties of which are incorporated herein by reference. In one or more instances, the selected catalyst may be combined or impregnated with a co-catalyst (e.g., a co-catalyst having hydrogenation/dehydrogenation functionality), a binder, and the like, and any combination thereof.

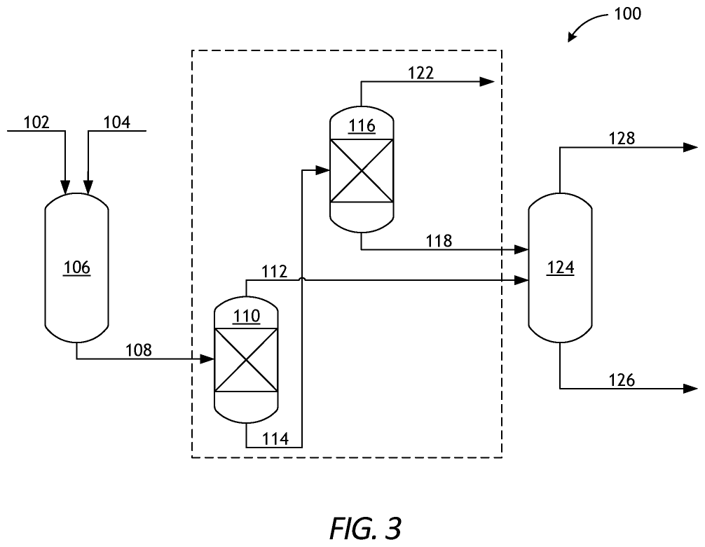

Referring now to FIG. 3, illustrated is a representative system 100 for implementing an isomerization method, according to one or more aspects of the present disclosure. Isomerization reactor 106 receives n-paraffin stream 102 comprised of a primary reactant (e.g., n-C7) and hydrogen stream 104 at a selected hydrogen:hydrocarbon molar ratio and LHSV. Isomerization reactor 106 is operated at a selected isomerization temperature and pressure parameter. The pressure parameter is determined based on total pressure, isomerization temperature, hydrogen:hydrocarbon ratio, and the molar ratio of the primary reactant in the n-paraffin stream 102; that is, the pressure parameter is selected based on the vapor pressure of the primary reactant in the n-paraffin stream 102 at the isomerization temperature, n-paraffin mol % in the feedstock and H2:hydrocarbon molar ratio, as described hereinabove (e.g., Equations 1-3 and Table 1). As the n-paraffin is converted, the pressure parameter also changes as it is based on the available n-paraffin.

For a batch process, the pressure parameter can be adjusted during the run to keep the partial pressure of n-paraffin close to the vapor pressure. For a continuous process, as shown in FIG. 3, multiple-stage reactors may be used. The output 108 of the isomerized n-paraffin may be fed into a separator 110 to remove hydrogen 112 therefrom, which may optionally be fed into a second isomerization reactor 124. The separated isomerization product 114 may be optionally fed into a secondary separator 116 for removal of the isomer product 122. Any remaining n-paraffin rich 118 may be fed into the second reactor 124 with the hydrogen stream 112 to produce final isomerization product 126 and hydrogen recycle product 128. The n-paraffin 118 can be concentrated due to its higher boiling point compared to isomer products. Moreover, interstage separator 116 allows a relatively lower pressure to be used in reactor 124. While only two reactors are shown in FIG. 3, it is to be appreciated that one or more than two reactors may be used, without departing from the scope of the present disclosure.

Further, while the present disclosure is described in terms of n-paraffin isomerization, the pressure parameter and methods described herein are equally applicable to other process types in which adsorption-reaction reactors are used.

The present disclosure provides, among others, the following aspects, each of which may be considered as optionally including any alternate thereof.

Clause 1: A method comprising: processing an isomerization feedstock comprising a primary n-paraffin reactant and hydrogen gas in an isomerization reactor comprising a catalyst, wherein the isomerization reactor is operated at a pressure parameter at which a partial pressure of the primary n-paraffin is within about 70% to about 130% of its equilibrium vapor pressure to isomerize the primary n-paraffin reactant.

Clause 2: The method according to Clause 1, wherein the primary n-paraffin reactant is n-C4, n-C5, n-C6, n-C7, or n-C8.

Clause 3: The method according to Clause 1 or Clause 2, wherein the primary n-paraffin reactant is present in an amount of greater than about 6% by weight of the feedstock.

Clause 4: The method according to any of the preceding Clauses, wherein the pressure parameter is in the range of about 70 psia to about 600 psia.

Clause 5: The method according to any of the preceding Clauses, wherein the isomerization reactor is operated at a temperature in the range of about 120° C. to about 220° C.

Clause 6: The method according to any of the preceding Clauses, wherein a molar ratio of hydrogen gas to total hydrocarbon in the feedstock is in the range of about 0.5 to about 4.0.

Clause 7: The method according to any of the preceding Clauses, wherein the isomerization reactor is operated at a liquid hourly space velocity in the range of about 0.5 hr⁻¹ to about 8.0 hr⁻¹.

Clause 8: The method according to any of the preceding Clauses, wherein the primary n-paraffin reactant is isomerized at an overall conversion rate in the range of about 10% to about 100% of an initial n-paraffin feedstock.

Clause 9: The method according to any of the preceding Clauses, wherein the catalyst is a metal selected from the group consisting of a Group IVB (Group 4), a Group VIB metal (Group 6), a Group VIIIB (Groups 8, 9, or 10), a zeolite, a molecular sieve or crystalline microporous material of the MWW framework type, and any combination thereof.

Clause 10: The method according to any of the preceding Clauses, wherein the catalyst is a mixed metal oxide catalyst of WOxZry catalyst.

Clause 11: A system comprising: an isomerization reactor comprising a catalyst for receiving an isomerization feedstock comprising a primary n-paraffin reactant and hydrogen gas, wherein the isomerization reactor is operated at a pressure parameter at which a partial pressure of the primary n-paraffin is within about 85% to about 115% of its equilibrium vapor pressure; and a first separator for separating isomerization product and recycled hydrogen gas.

Clause 12: The system according to Clause 11, wherein the primary n-paraffin reactant is n-C4, n-C5, n-C6, n-C7, or n-C8.

Clause 13: The system according to Clause 11 or Clause 12, wherein the primary n-paraffin reactant is present in an amount of greater than about 6% by weight of the feedstock.

Clause 14: The system according to any of Clause 12 to Clause 13, wherein the pressure parameter is in the range of about 70 psia to about 600 psia.

Clause 15: The system according to any of Clause 12 to Clause 14, wherein the isomerization reactor is operated at a temperature in the range of about 120° C. to about 220° C.

Clause 16: The system according to any of Clause 12 to Clause 15, wherein a molar ratio of hydrogen gas to total hydrocarbon reactant in the feedstock is in the range of about 0.5 to about 4.0.

Clause 17: The system according to any of Clause 12 to Clause 16, wherein the isomerization reactor is operated at a liquid hourly space velocity in the range of about 0.5 hr⁻¹ to about 8.0 hr⁻¹.

Clause 18: The system according to any of Clause 12 to Clause 17, wherein the primary n-paraffin reactant is isomerized at an overall conversion rate in the range of about 10% to about 100% of an initial n-paraffin feedstock.

Clause 19: The system according to any of Clause 12 to Clause 18, wherein the catalyst is a metal selected from the group consisting of a Group IVB (Group 4), a Group VIB metal (Group 6), a Group VIIIB (Groups 8, 9, or 10), a zeolite, a molecular sieve or crystalline microporous material of the MWW framework type, and any combination thereof.

Clause 20: The system according to any of Clause 12 to Clause 19, wherein the catalyst is a mixed metal oxide catalyst of WOxZry catalyst.

To facilitate a better understanding of one or more aspects of the present disclosure, the following example is given. In no way should the following example be read to limit, or to define, the scope of the disclosure. Indeed, the example below is merely illustrative of various aspects of the present disclosure and should not be considered limiting in any way.

### EXAMPLE

### n-Heptane Isomerization Test

In this example, pure n-heptane (n-C7) feedstock was isomerized in the presence of hydrogen using multiple pilot runs to evaluate process conditions. In an isomerization reactor, a fix catalyst bed of platinum impregnated (WO3)x/(ZrO2)y was used. The isomerization reaction was via an adsorption-reaction mechanism. The feedstock was pumped through the catalyst bed in the reactor held at a reaction temperature of 180° C., a LHSV of 2 hr⁻¹, a hydrogen:hydrocarbon molar ratio of 1.5, and an initial partial pressure of n-C7 between 20 psia and 240 psia to determine the influence of partial pressure on the overall n-C7 conversion rate, corresponding to the overall reaction rate. In total, 114 pilot runs with five different initial n-C7 partial pressures (25 psia, 60 psia, 105 psia, 150 psia, and 210 psia) were included. Each of the multiple pilot runs had identical residence times, as LHSV remained the same for each. The products of the isomerization processes were analyzed using online gas chromatography (GC).

The overall conversion value of n-C7 (Conv(nC₇)) is defined in the following Equation:

$$Conv ( n {C}_{7} ) = \frac{Δ m ( n {C}_{7} )}{m ( n {C}_{7}^{0} )}$$

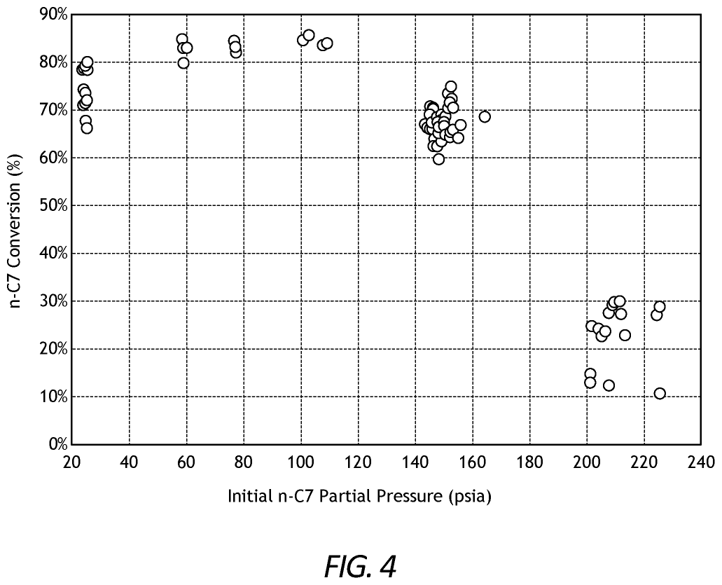

FIG. 4 provides a plot showing the overall n-C7 conversion against the initial partial pressures tested in this Example (representing 114 separate runs), in which initial partial pressure is provided in psia along the x-axis and overall conversion is provided in % of initial feedstock along the y-axis. For runs performed at the same initial partial pressure, the average overall n-C7 conversion value at that partial pressure was used.

As shown in FIG. 4, n-C7 overall conversion rate increases with pressure when the pressure is low, reaches a peak value, and the proceeds to decrease as pressure increases. As provided above, surprisingly, conventional understanding in the industry is that isomerization performed at higher pressures prompts increased conversion rate. However, as shown in FIG. 4, relatively low isomerization pressures actually increase conversion rate. With continued reference to FIG. 4, peak conversion occurred at a pressure of about 105 psia, corresponding to the approximate vapor pressure of n-C7 at 180° C. (see FIG. 1). The C7 isomer distributions (%) of the total isomerization product at each pressure of this Example are listed in Table 2.

TABLE 2

| Initial n-C7 | | | | | |
|---|---|---|---|---|---|
| Partial | | | Mono-Methyls- | Di-Methyls | Tri-methyl- |
| Pressure | | n-C7 | Hexane | Pentane | butane |
| 25 | psia | 33.3% | 51.9% | 13.0% | 1.7% |
| 60 | psia | 22.8% | 57.9% | 16.8% | 2.5% |
| 105 | psia | 20.3% | 61.5% | 15.9% | 2.3% |
| 150 | psia | 37.9% | 49.5% | 11.4% | 1.2% |
| 210 | psia | 77.8% | 17.9% | 4.1% | 0.2% |

Without being bound by theory, as described above, it is believed that the n-C7 isomerization rate peaked at or near the pressure at which the initial partial pressure of n-C7 is close to its vapor pressure (gas-liquid transition phase) based on either or both of: (1) at or near the gas-liquid transition phase, formation of n-C7 reactant on or about the catalyst surface was promoted while maintaining diffusion lower than the condensed liquid phase, thus promoting overall adsorption of the n-C7 reactant on catalyst active sites and/or (2) the boiling status on the catalyst surface provided an effective way for n-C7 isomer product desorption from the catalyst surface.

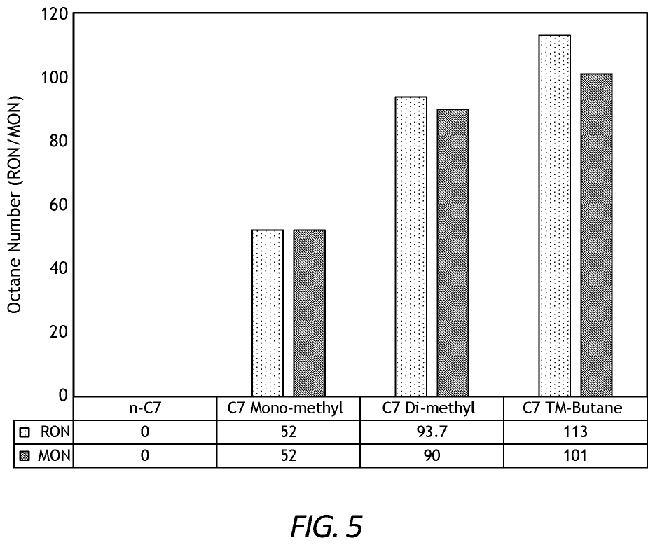

Isomerization products with high number of branches have higher octane numbers, as shown in FIG. 5. Octane numbers for these representative isomers were the average of measured Research Octane Number (RON) and Motor Octane Number (MON). RON was determined according to ASTM D2699 and MON was determined according to ASTM D2700. FIG. 5 shows that (1) mono-methyl C7 paraffins, such as 2-methylhexane and 3-methyl hexane, have RON and MON at 52, compared to n-C7, which has RON and MON at 0; (2) di-methyl C7 paraffins, such as 2,2-dimethyl pentane, 2,3-dimethyl pentane, 2,4-dimethyl pentane, and 3,3-dimethyl pentane, have RON and MON at 93.7 and 90, respectively; and (3) tri-methyl C7 paraffin, such as 2,2,3-trimethyl butane, has RON and MON at 113 and 101, respectively. Higher conversion of n-heptane and high selectivity of high-branched isomers will have a high octane number for the product mixture.

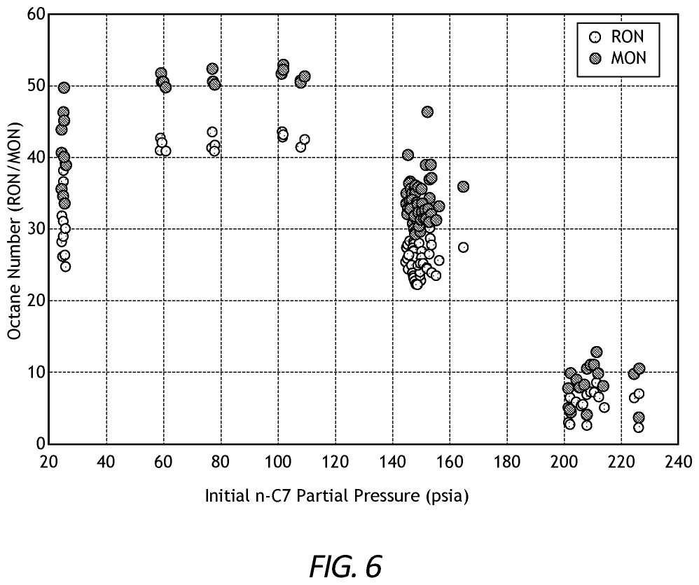

The product compositions in the pilot runs of this example were measured using GC method. The compositions were used for Octane number calculation using the Octane model developed in-house, as described in Ghosh, P. et al. “Development of a Detailed Gasoline Composition-Based Octane Model,” Industrial & Engineering Chemistry Research 45 (2006): 337-345, incorporated herein in its entirety. The plot of product RON and MON vs. initial n-C7 pressure of the instant Example is shown in FIG. 6. The feedstock n-C7 had both RON and MON at 0. Higher conversion of n-C7 to iso-C7 will have a high product Octane number, as shown in FIG. 6.

Any documents described herein are incorporated by reference herein for purposes of all jurisdictions where such practice is allowed, including any priority documents and/or testing procedures to the extent they are not inconsistent with this text. As is apparent from the foregoing general description and the specific aspects described herein, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the various aspects of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed, including the lower limit and upper limit. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

Therefore, the present disclosure is well adapted to attain the ends and advantages mentioned as well as those that are inherent therein. The particular aspects disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative aspects disclosed above may be altered, combined, or modified and all such variations are considered within the scope and spirit of the present disclosure. The aspects illustratively disclosed herein suitably may be practiced in the absence of any element that is not specifically disclosed herein and/or any optional element disclosed herein.

What is claimed is:

1. A method comprising:
processing an isomerization feedstock comprising a primary n-paraffin reactant and hydrogen gas in an isomerization reactor comprising a catalyst, wherein the isomerization reactor is operated at a pressure parameter at which a partial pressure of the primary n-paraffin is within about 70% to about 130% of its equilibrium vapor pressure to isomerize the primary n-paraffin reactant.

2. The method of claim 1, wherein the primary n-paraffin reactant is n-C4, n-C5, n-C6, n-C7, or n-C8.

3. The method of claim 1, wherein the primary n-paraffin reactant is present in an amount of greater than about 6% by weight of the feedstock.

4. The method of claim 1, wherein the pressure parameter is in the range of about 70 psia to about 600 psia.

5. The method of claim 1, wherein the isomerization reactor is operated at a temperature in the range of about 120° C. to about 220° C.

6. The method of claim 1, wherein a molar ratio of hydrogen gas to total hydrocarbon in the feedstock is in the range of about 0.5 to about 4.0.

7. The method of claim 1, wherein the isomerization reactor is operated at a liquid hourly space velocity in the range of about 0.5 hr⁻¹ to about 8.0 hr⁻¹.

8. The method of claim 1, wherein the primary n-paraffin reactant is isomerized at an overall conversion rate in the range of about 10% to about 100% of an initial n-paraffin feedstock.

9. The method of claim 1, wherein the catalyst is a metal selected from the group consisting of a Group IVB (Group 4), a Group VIB metal (Group 6), a Group VIIIB (Groups 8, 9, or 10), a zeolite, a molecular sieve or crystalline microporous material of the MWW framework type, and any combination thereof.

10. The method of claim 1, wherein the catalyst is a mixed metal oxide catalyst of WOxZry catalyst.

11. A system comprising:
an isomerization reactor comprising a catalyst for receiving an isomerization feedstock comprising a primary n-paraffin reactant and hydrogen gas, wherein the isomerization reactor is operated at a pressure parameter at which a partial pressure of the primary n-paraffin is within about 85% to about 115% of its equilibrium vapor pressure; and
a first separator for separating isomerization product and recycled hydrogen gas.

12. The system of claim 1, wherein the primary n-paraffin reactant is n-C4, n-C5, n-C6, n-C7, or n-C8.

13. The system of claim 1, wherein the primary n-paraffin reactant is present in an amount of greater than about 6% by weight of the feedstock.

14. The system of claim 1, wherein the pressure parameter is in the range of about 70 psia to about 600 psia.

15. The system of claim 1, wherein the isomerization reactor is operated at a temperature in the range of about 120° C. to about 220° C.

16. The system of claim 1, wherein a molar ratio of hydrogen gas to total hydrocarbon reactant in the feedstock is in the range of about 0.5 to about 4.0.

17. The system of claim 1, wherein the isomerization reactor is operated at a liquid hourly space velocity in the range of about 0.5 hr⁻¹ to about 8.0 hr⁻¹.

18. The system of claim 1, wherein the primary n-paraffin reactant is isomerized at an overall conversion rate in the range of about 10% to about 100% of an initial n-paraffin feedstock.

19. The system of claim 1, wherein the catalyst is a metal selected from the group consisting of a Group IVB (Group 4), a Group VIB metal (Group 6), a Group VIIIB (Groups 8, 9, or 10), a zeolite, a molecular sieve or crystalline microporous material of the MWW framework type, and any combination thereof.

20. The system of claim 1, wherein the catalyst is a mixed metal oxide catalyst of WOxZry catalyst.
